(19) United States
(12) Patent Application Publication
(10) Pub. No.: US 2008/0161904 A1
(43) Pub. Date: Jul. 3, 2008

(54) CONNECTION OF ADJACENT BLOOD VESSELS

(76) Inventors: Richard R. Heuser, Phoenix, AZ (US); James D. Joye, Saratoga, CA (US)

Correspondence Address: KOLISCH HARTWELL, P.C., 520 SW YAMHILL STREET, Suite 200, PORTLAND, OR, 97204, US

(21) Appl. No.: 12/050,799

(22) Filed: Mar. 18, 2008

Related U.S. Application Data
(63) Continuation-in-part of application No. 11/340,324, filed on Jan. 25, 2006, now Pat. No. 7,374,567.

(51) Int. Cl. A61F 2/06

(52) U.S. Cl. 623/1.13; 623/1.23; 623/1.1

(57) ABSTRACT

A catheter apparatus, tissue-engineered vessel and a method of using the tissue-engineered vessel are provided for the connection of two adjacent blood vessels. In some embodiments, the tissue engineered vessel may be affixed to a stent, or a stent may be inserted into an area to be treated first, and then the tissue-engineered vessel may be affixed to the stent. The tissue-engineered vessel may include a living adventitia, a decellularized internal membrane, and/or an endothelium.

### CROSS REFERENCE TO RELATED APPLICATIONS

This application is a continuation-in-part of application Ser. No. 11/340,324, filed Jan. 25, 2006, the disclosure of which is incorporated by reference.

### BACKGROUND

A catheter apparatus and method for arterializing a section of a vein to bypass a clogged artery are shown in U.S. Pat. No. 6,464,665, which is hereby incorporated by reference. The method is used to bypass a stenosis in the artery that obstructs blood flow in a portion of the artery. If the obstructed portion of the artery can be bypassed, blood flow will be restored downstream from the stenosis. A vein running alongside the artery in the obstructed portion of the artery can be used for the bypass.

The catheter apparatus includes one catheter for inserting into the artery and another catheter for inserting into the adjacent vein. The physician maneuvers the tips of both catheters to coincident positions within each blood vessel adjacent one end of the obstructed portion of the artery. The physician then creates an opening from the inside of one blood vessel through the vessel wall and then through the wall of the other blood vessel. A difficulty here is in co-locating the openings in the two blood vessels and holding the vessel walls in place to ensure that a channel will be created between the vessels so that blood will flow from one vessel to the other.

### SUMMARY

A catheter apparatus, tissue-engineered vessel and a method of using the tissue-engineered vessel are provided for the connection of two adjacent blood vessels. In some embodiments, the tissue-engineered vessel may be affixed to a stent, or a stent may be inserted into an area to be treated first, and then the tissue-engineered vessel may be affixed to the stent. The tissue-engineered vessel may include a living adventitia, a decellularized internal membrane, and/or an endothelium.

### BRIEF DESCRIPTION OF THE DRAWINGS

FIG. 1 is a partial cross-sectional view showing an obstructed artery, including the obstruction and the area adjacent both ends of the obstruction, and a vein alongside the artery.

FIG. 2 is a cross-sectional view of an embodiment in the blood vessels of FIG. 1 with a first catheter with a distal end inserted into the artery and a second catheter with a distal end inserted into the vein, the catheters carrying at their distal ends mating tips, i.e., a piercing tool on the first catheter and a receptor on the second catheter.

FIG. 3 is a cross-sectional view of the vein, artery, and two catheters, as in FIG. 2 with the tips of the catheters mated to create a pair of co-located openings in the walls of the vein and artery for connection of a fistula between the artery and the vein.

FIG. 4 is a cross-sectional view of the vein and artery with a balloon inserted through both openings.

FIG. 5 is a cross-sectional view of the vein and artery with a device, such as a stent or tissue-engineered vessel, installed through the openings between the vein and artery to maintain a fistula therebetween.

FIG. 6 is a cross-sectional view of a first catheter inserted in the artery and a second catheter inserted in the vein at the other end of the obstruction depicted in FIGS. 1-4, the catheters including mating tips shown in a joined position to create a second pair of co-located openings through the vein and artery walls.

FIG. 7 is a cross-sectional view of the vein and artery with a balloon inserted through the second pair of openings between the vein and the artery.

FIG. 8 is a cross-sectional view of the vein and artery with a second device, such as a stent or tissue-engineered vessel, installed through the second pair of openings between the vein and artery to maintain a fistula therebetween.

FIG. 9 is a close-up perspective view of the mating tips of the first and second catheters, showing the receptor, which includes a proximal end, a distal opening, and a channel providing a guide surface, and the piercing tool, which includes a needle and a plug encompassing the catheter adjacent the base of the needle, and showing the contours of the plug, needle, and receptor channel that provide for mating between the tips.

FIG. 10 is a piercing tool for use in a second embodiment that includes a base and a needle that is offset from the base by an angle.

FIG. 11 illustrates the use of the piercing tool of FIG. 10 in conjunction with a double-balloon catheter to create openings in a vein and an artery.

FIG. 12 illustrates the use of the piercing tool of FIGS. 2, 3, 6, and 9 in conjunction with a double-balloon catheter to create openings in a vein and an artery.

### DETAILED DESCRIPTION OF THE PREFERRED EMBODIMENTS

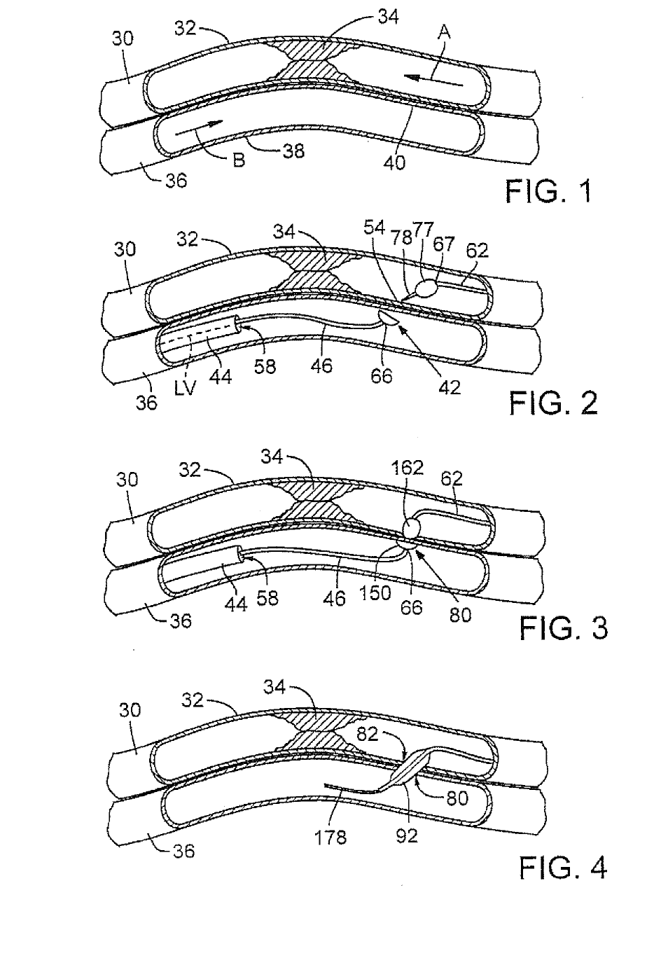

As shown in FIG. 1, an artery 30, formed by an artery wall 32, has a blood flow, indicated by arrow A, that is partially or totally blocked by an obstruction or occlusion 34, typically formed by plaque. A vein 36 roughly similar in dimension to artery 30 lies alongside and generally parallel to artery 30. Vein 36, formed by a vein wall 38, includes, in the area proximal to occlusion 34, a portion 40 in close proximity to artery 30 that the physician has selected as a venous site for creating a fistula between artery 30 and vein 36. The normal blood flow through vein 36 would be in the direction indicated by arrow B.

An embodiment of the invented system, indicated generally at 42 in FIG. 2, is a catheter apparatus that includes a first catheter 62 and a second catheter 44. In FIG. 2, the first catheter is in the artery and the second catheter is in the vein, but this can be reversed. Similarly, the first catheter in the artery is shown upstream from occlusion 34, but this may alternatively be reversed to begin the procedure downstream from the occlusion and proceeding afterwards to the upstream side.

Second catheter 44 may include at least one lumen 58 which runs generally parallel to a longitudinal axis LV of catheter 44. A wire 46 may be inserted through lumen 58. Typically, wire 46 has an outer diameter of 0.035-inches, but any suitable dimension may be used. Wire 46 may be controllable by the physician in position relative to catheter 44. Wire 46 may be a guidewire for catheter 44, or a separate guidewire may be used, with other lumens in catheter 44 providing the channel for the separate guidewire.

As shown in FIG. 2, first catheter 62 of catheter apparatus 42 includes a distal end 67 that the physician may insert into artery 30 for positioning adjacent arterial fistula site 54. First catheter 62 may include one or more lumens running generally parallel to a longitudinal axis of catheter 62. First catheter 62 may be guided along a guidewire or may itself be a guidewire, typically with an outer diameter of 0.035-inches, although any suitable dimension may be used. First catheter 62 preferably is hollow.

A piercing tool 77 that includes a sharp needle 78 may be selectively deployed, as shown in FIGS. 2 and 3, or withdrawn into the lumen of catheter 62. Needle 78 is preferably withdrawn while catheter 44 is maneuvered to the fistula site so as not to cause trauma to the blood vessel wall.

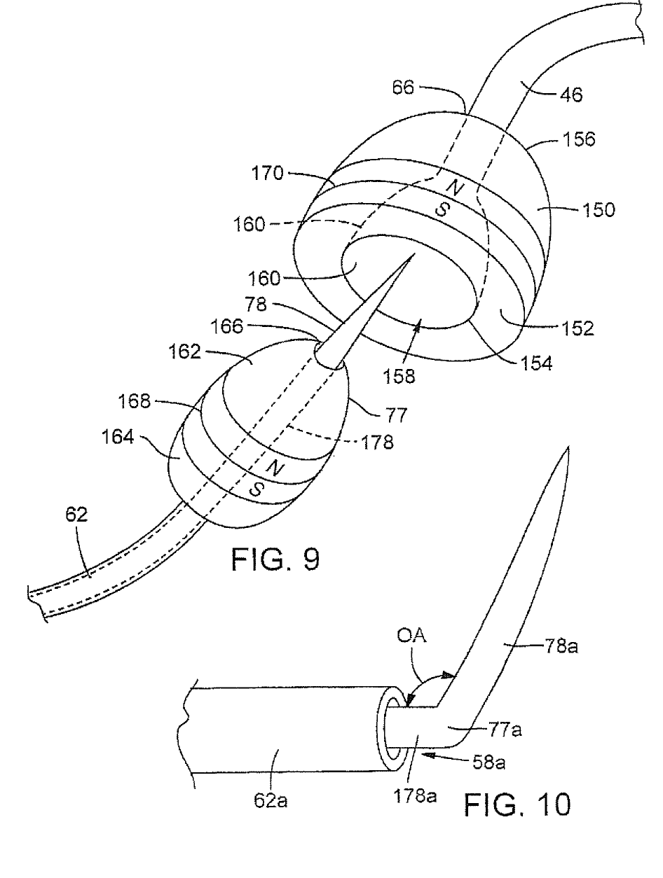

As best seen in FIG. 9, needle 78 may be disposed at the distal end of a wire 178 disposed in the lumen of catheter 62. The physician can control the positioning of wire 178 and needle 78 relative to catheter 62. Guidewire 46 may include a receptor 150, such as substantially cup-shaped socket 152. Receptor 150 includes a distal opening 154, preferably circular, and a proximal end 156. Receptor 150 includes a channel 158 leading from opening 154 toward proximal end 156. Channel 158 preferably narrows in a direction from opening 154 toward proximal end 156. Channel 158 is defined by an inner surface 160 that provides a guide surface for needle 78 that directs the needle toward proximal end 156 of receptor 150. Channel 158 may be substantially conical, or have such other shape as tends to mate with, and guide piercing tool 77 into receptor 150.

Piercing tool 77 on catheter 62 preferably includes a plug 162 provided with an outer contour that narrows from a proximal end 164 toward a distal end 166. Plug 162 preferably mates with channel 158 in receptor 150. Plug 162 preferably encompasses catheter 62 adjacent the distal end of the catheter. As seen in FIGS. 2, 3, and 9, the piercing tool and the receptor have a complementary configuration that supports their mating together.

Typically, piercing tool 77 will include a magnet with one pole oriented toward the distal end of the tool, while receptor 150 will include a magnet with the opposite pole oriented toward the distal end of the receptor which will draw the needle into the receptor. For example, the magnets may be annular rings or donuts and formed of a strong permanent magnet material suitable for the intended use.

A typical arrangement, shown in FIG. 9, is that plug 162 includes a first magnet 168 generally in a donut shape and having a north pole N positioned distally with respect to a south pole S. Typically magnet 168 is spaced from the distal end of plug 162. A second magnet 170 may be disposed on, or form an integral part of receptor 152, preferably adjacent distal opening 154 of socket 152. Second magnet 170 may be arranged with a south pole S distal of a north pole N to attract magnet 168 when the tips of the two catheters are in proximity, e.g., with each catheter in an adjacent blood vessel. Alternatively or in addition one or more magnets may be arranged in various locations on plug 162 and/or needle 78 and on or in receptor 150, e.g., adjacent proximal end 156, with the poles arranged to draw piercing tool 77 into receptor 150.

As shown in FIGS. 3 and 4, after creating openings 80, 82 with a tool such as needle 78, the physician withdraws catheter 62 from the fistula site, leaving wire 178 in place, and a balloon 92 may be inserted over wire 178 and through openings 80, 82 and inflated to enlarge the openings. Balloon 92 may include radiopaque markers and may be inflated with a solution containing a radiopaque dye or contrast to allow the physician to radiographically monitor and adjust the position of the balloon before, during, and after inflation.

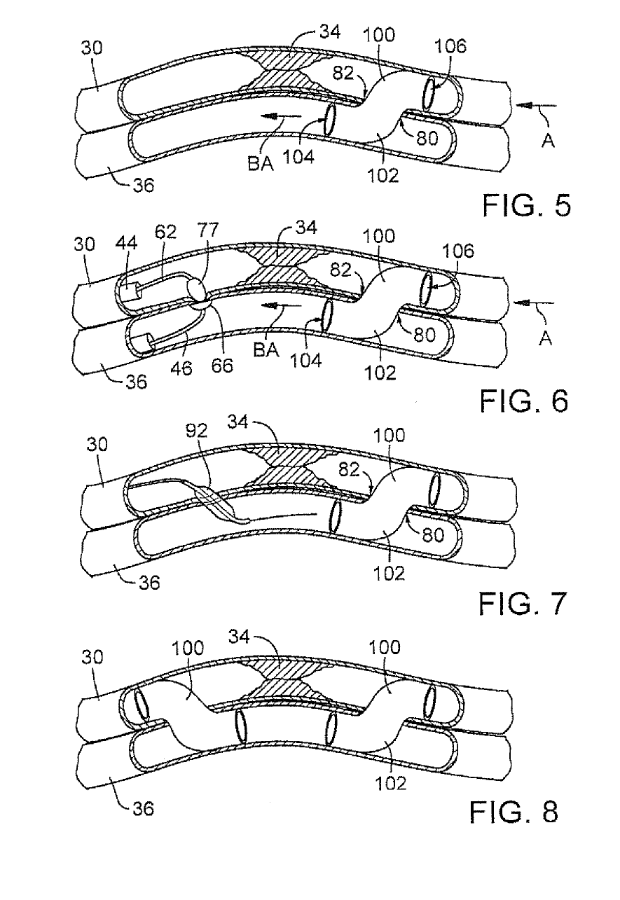

As shown in FIG. 5, a device 100 for maintaining an open, leak-free connection between openings 80 and 82, such as a stent or a tissue-engineered vessel, is inserted through the openings. Device 100 includes a wall 102 having two open ends 104 and 106 that preferably create leak-free couplings to the inside of artery 30 and vein 36. With openings 80, 82 connected to form a fistula, vein 36 is arterialized, and blood flows from artery 30 into vein 36 in the direction indicated by arrows A and BA.

In some embodiments, device 100 may be a short, covered stent, such as the Hemobahn stent made by WL Gore & Associates.

In other embodiments, device 100 may include a tissue-engineered vessel. Such a vessel may be created with the use of autologous cells. Autologous cells may include any type of cell found in a living body, such as fibroblasts and endothelial cells. Autologous cells may be harvested from a biopsy specimen of skin, vein, artery, internal organ, or any other component of a living body.

Tissue-engineered vessels may be created using various techniques, including sheet-based tissue engineering. This technique is described in detail in “Human Tissue-Engineered Blood Vessels for Adult Arterial Revascularization,” Nature Medicine 2006;12:361-5, published Feb. 19, 2006, which is incorporated by reference in its entirety for all purposes. Tissue-engineered vessels may include one or more of the following components: a living adventitia, a decellularized internal membrane, and an endothelium, each forming a layer of the vessel.

The internal membrane may provide a barrier against cell migration toward the lumen of the tissue-engineered vessel. It may be created by wrapping a fibroblast sheet around a support tube for one or more revolutions. After a predetermined maturation period, which in some embodiments may be at least 10 weeks, the plies of each revolution may fuse together for form a homogenous cylindrical tissue. In some embodiments, this homogenous cylindrical tissue may be dehydrated to form an acellular substrate for endothelial cell seeding.

Tissue-engineered vessels may be configured to fuse with the outer or inner walls of the artery 30 or vein 36 adjacent to openings 80, 82, creating a seal with the vessels which prevents leakage. In addition to integration between the tissue-engineered vessel and the artery 30 and/or vein 36, some tissue-engineered vessels may be advantageously configured to form vasa vasorum for providing oxygen and nutrients to the tissue-engineered vessel.

In some embodiments, device 100 may include a combination of a stent and tissue-engineered vessel. For instance, a stent may be deposited first. Next, a tissue-engineered vessel may be installed within the stent. The tissue-engineered vessel may be configured to affix to the stent, for example by grafting, or it may be configured to remain separate from the stent, so that the stent may be removed once the tissue-engineered vessel is secure. In other embodiments, the tissue-engineered vessel may already be affixed to the stent prior to the stent being deposited.

As shown in FIGS. 6, 7, and 8 a second pair of co-located openings may be created, and a stented fistula established therebetween, using essentially the same catheter system and method as described for FIGS. 1-5 and 9. FIG. 6 illustrates that the first catheter with the piercing tool preferably is inserted into the artery and the openings created from the artery into the vein. Alternatively the openings may be created from the vein into the artery.

An alternative embodiment for the piercing tool in shown in FIG. 10. This tool 77a may be used with a metal guidewire 62a that preferably includes a lumen 58a. An inner wire 178a may be inserted in lumen 58a, providing a base for a needle 78a. The coupling between the needle and base incorporates a curvature such that the needle is nominally offset from the base by an angle OA, typically between about 30-degrees and about 90-degrees. Inner wire 178a is typically made of a sufficiently rigid material, such as nitinol and/or stainless steel, as to maintain the offset angle as the needle is used to pierce blood vessels. Guidewire 62a is preferably formed of a sufficiently rigid material such that when needle 78a is retracted into lumen 58a, the curvature between the needle and the base is overcome and the needle temporarily aligns with the base in a non-traumatic configuration. Inner wire 178a may have an outer diameter of 0.010, 0.014, 0.018, or 0.021-inches, or such other dimension as is suited to the particular application.

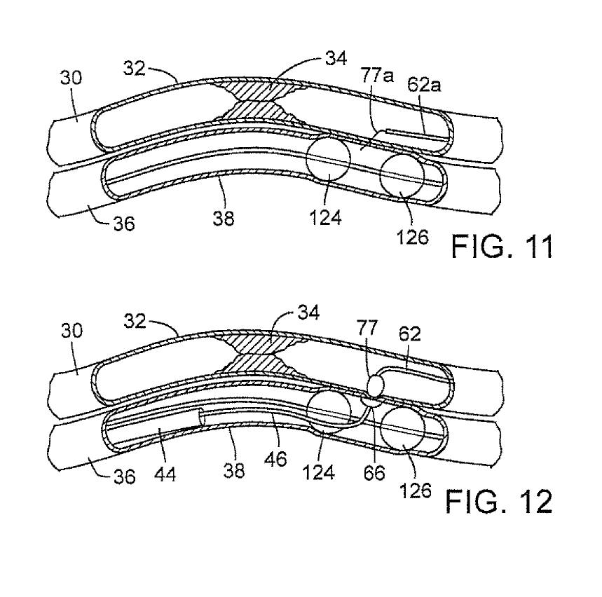

As shown in FIG. 11, piercing tool 77a may be inserted in artery 30, typically while withdrawn into the catheter 62a while maneuvering to the fistula site. Piercing tool 77a may be used in conjunction with a catheter having two balloons 124 and 126 that are inserted in vein 36. In such case, the catheter tips are maneuvered to opposing sides of the proposed fistula site and balloons 124 and 126 are inflated to press the vein wall against the artery wall. Also, fluid may be injected into the sealed-off area to further press the two blood vessel walls together. Then piercing tool 77a is deployed and maneuvered through the artery and then the vein wall to create openings for forming the fistula as for the embodiments described above.

FIG. 11 depicts the piercing tool and the balloon catheter in different vessels. Alternatively, piercing tool 77a may be inserted in the same blood vessel with the balloon catheter. In such an embodiment, the balloons are preferably independently inflatable, and typically the distal balloon 124 is inflated first to stop blood flow. Then, piercing tool 77a is maneuvered to the fistula site in a manner similar to that for the previously described embodiment, typically with the piercing tool withdrawn into the guidewire to the non-traumatic configuration.

With the piercing tool at the fistula site, the proximal balloon 126 is inflated to seal off the fistula site and also to press the vein against the artery. Then, piercing tool 77a is deployed at the end of guidewire 62a and maneuvered by the physician to create the openings from one blood vessel, through both walls, to the other blood vessel.

In either case, piercing tool 77a may be used to create multiple pairs of co-located openings which are then stented to arterialize a portion of the vein to bypass a blockage using a similar method as described above for the embodiment of FIGS. 1-9.

As shown in FIG. 12, the double balloon catheter may also be used in conjunction with the catheters 44 and 62 that include the mating tips. In this embodiment, the double balloon catheter helps to control blood flow at the planned fistula site and to press the blood vessel walls together to assist in the mating of the tips. The fistula creation otherwise proceeds in a similar manner as for the embodiment of FIGS. 1-9.

It is believed that the disclosure set forth above encompasses multiple distinct embodiments with independent utility. While each of these embodiments has been disclosed in its preferred form, the specific embodiments thereof as disclosed and illustrated herein are not to be considered in a limiting sense as numerous variations are possible. The subject matter of the embodiments includes all novel and non-obvious combinations and subcombinations of the various elements, features, functions and/or properties disclosed herein. No single feature, function, element or property of the disclosed embodiments is essential to all of the disclosed embodiments. Similarly, where the claims recite “a” or “a first” element or the equivalent thereof, such claims should be understood to include incorporation of one or more such elements, neither requiring nor excluding two or more such elements.

It is believed that the following claims particularly point out certain combinations and subcombinations that are directed to one of the disclosed embodiments and are novel and non-obvious. Inventions embodied in other combinations and subcombinations of features, functions, elements and/or properties may be claimed through amendment of the present claims or presentation of new claims in this or a related application. Such amended or new claims, whether they are directed to a different embodiment or directed to the same embodiment, whether different, broader, narrower or equal in scope to the original claims, are also included within the subject matter of the embodiments of the present disclosure.

I claim:

1. A catheter apparatus for arterializing a vein by creating a fistula between the vein and an artery, the apparatus comprising:
an arterial catheter having a first distal end insertable to a position wherein the first distal end is adjacent a site within the artery for the fistula;
a venous catheter having a second distal end insertable to a position wherein the second distal end is adjacent a site within the vein for the fistula;
a tool for creating an opening through the wall of the artery adjacent the arterial fistula site and an opening through the wall of the vein adjacent the venous fistula site; and
a tissue-engineered vessel for connecting the artery and the vein at the fistula sites, the tissue-engineered vessel including an inner passageway connecting a first open end configured to be disposed in the artery and a second open end configured to be disposed in the vein.

2. The catheter apparatus of claim 1 wherein the tissue-engineered vessel for connecting the artery and the vein at the fistula sites includes any of a living adventitia, a decellularized internal membrane, and an endothelium.

3. The catheter apparatus of claim 1 further comprising a stent for insertion to a position between the artery and the vein at the fistula sites, the stent being configured to be received the tissue-engineered vessel.

4. A vessel for connecting an artery and a vein through an opening in the artery wall and an opening in the vein wall, the vessel comprising:
a first open end,
a second open end, and
a passageway therebetween,
the passageway defining a cross-sectional area, the vessel being tissue-engineered.

5. The vessel of claim 4 further comprising any of a living adventitia, a decellularized internal membrane and an endothelium.

6. The vessel of claim 4 further comprising a stent to which the vessel is affixed.

7. A method of arterializing a peripheral vein by creating a fistula between the vein and a peripheral artery, the method comprising:
providing an arterial catheter having a distal end and inserting the arterial catheter into the artery to a position wherein the distal end is adjacent a site for the fistula within the artery;
providing a venous catheter having a distal end and inserting the venous catheter into the vein to a position wherein the distal end is adjacent a site for the fistula within the artery;
creating an opening through the wall of the artery adjacent the arterial fistula site and creating an opening through the wall of the vein adjacent the venous fistula site;
providing a tissue-engineered vessel having a first open end and a second open end; and
after creating openings in the vein and artery, depositing the tissue-engineered vessel through the openings in the artery and the vein with the first open end disposed in the artery and the second open end disposed in the vein.

8. The method of claim 7 wherein the tissue-engineered vessel includes any of a living adventitia, a decellularized internal membrane and an endothelium.

9. The method of claim 7 further including the steps of providing a tubular stent with a third open end and a fourth open end, and, after creating the openings in the vein and artery, depositing the stent through the openings in the artery and the vein with the third open end disposed in the artery and the fourth open end disposed in the vein, and affixing the tissue-engineered vessel to the stent.

10. The method of claim 7 wherein the tissue-engineered vessel is provided affixed to a tubular stent.
